(19) United States
(12) Patent Application Publication
(10) Pub. No.: US 2013/0243867 A1
(43) Pub. Date: Sep. 19, 2013

(54) MICELLE COMPOSITIONS AND METHODS FOR THEIR USE

(71) Applicants: Subhra Mohapatra, Tampa, FL (US); Shyam S. Mohapatra, Tampa, FL (US); Mark Christian Howell, Tampa, FL (US); Suraj Kishore Dixit, Tampa, FL (US); Chunyan Wang, Tampa, FL (US)

(72) Inventors: Subhra Mohapatra, Tampa, FL (US); Shyam S. Mohapatra, Tampa, FL (US); Mark Christian Howell, Tampa, FL (US); Suraj Kishore Dixit, Tampa, FL (US); Chunyan Wang, Tampa, FL (US)

(73) Assignee: University of South Florida (A Florida Non-Profit Corporation), Tampa, FL (US)

(21) Appl. No.: 13/773,749

(22) Filed: Feb. 22, 2013

Related U.S. Application Data
(60) Provisional application No. 61/602,384, filed on Feb. 23, 2012.

(51) Int. Cl. A61K 9/16; A61K 31/7088

(52) U.S. Cl. CPC A61K 9/167; A61K 31/7088
USPC 424/493; 514/44 R

(57) ABSTRACT

Provided herein is a micelle composition comprising a polyethylene glycol (PEG), a DC-cholesterol, and a dioleoylphosphatidyl-ethanolamine (DOPE) and either or both a pharmaceutical compound core and a polynucleotide coating. Also provided herein is a method of administering one or more compounds to a cell comprising administering to the cell a micelle composition comprising 1) PEG-PE, a DC-cholesterol, and DOPE, and 2) the one or more compounds, wherein the compounds are selected from the group consisting of a polynucleotide and a pharmaceutical composition. Further provided are methods for detecting the micelle composition.

### CROSS-REFERENCE TO RELATED APPLICATIONS

This application claims the priority benefit of U.S. Provisional Patent Application Ser. No. 61/602,384 filed on Feb. 23, 2012.

### STATEMENT AS TO RIGHTS TO INVENTIONS MADE UNDER FEDERALLY SPONSORED RESEARCH AND DEVELOPMENT

This invention was made with government support under grants 1R41CA139785 and 5R01CA152005-01 from the National Institutes of Health. The U.S. government has certain rights in this invention.

### BACKGROUND

Advances in nanoparticle technology have allowed the development of multifunctional nanoparticles for cancer detection, therapy, and treatment monitoring. Their numerous advantages include cell-targeted delivery to minimize the amount of drug needed to achieve a therapeutic dose [M. Schmitt-Sody et al., Clinical Cancer Research: An Official Journal of the American Association for Cancer Research 2003 9, 2335], increased bioavailability especially for hydrophobic drugs, reduced drug toxicity [X. H. Peng et al., ACS Nano 2011, 5, 9480], enhanced mucosal delivery that decreases first-pass metabolism [J. C. Sung, B. L. Pulliam, & D. A. Edwards, Trends in Biotechnology 2007, 25, 563], controllable timing of drug delivery (slow-sustained, pulsatile or stimulus-responsive) [I. Kim et al., Biomaterials 2012, 33, 5574; X. Shuai et al., Journal of Controlled Release: Official Journal of the Controlled Release Society 2004, 98, 415; H. Meng et al., ACS Nano 2011, 5, 4131], and the capacity to combine drugs and imaging agents in the same particle [M. M. Yallapu et al., Pharmaceutical Research 2010, 27, 2283; R. Kumar et al., Theranostics 2012, 714; J. Shin et al., Angew Chem Int Ed Engl 2009, 48, 321]. Scalability, safety, and cost remain the most formidable challenges in taking multifunctional nanoparticles from the bench to clinical trials.

Magnetic resonance imaging (MRI) is one of the most widely used noninvasive imaging and diagnostic techniques. It provides detailed anatomical images of the body and is excellent for imaging soft tissues. Contrast agents work by altering the T1, T2, or T1/T2 relaxation times of nearby protons. Positive contrast agents appear brighter on the MRI owing to an increase in T1 signal intensity caused by a reduction in the T1 relaxation times [E. C. Cho et al., Trends in Molecular Medicine 2010, 16, 561]. Superparamagnetic iron oxide nanoparticles have been extensively studied for use in T2 contrast imaging in conjunction with a diverse array of nanotherapeutics [Y. Ling et al., Biomaterials 2011, 32, 7139; S. Laurent et al., Chemical Reviews 2008, 108, 2064; R. Rastogi et al., Colloids and Surfaces B, Biointerfaces 2011, 82, 160]. Previously, we reported a unique formulation of chitosan-polyethyleneimine nanoparticles with iron oxide in the core for imaging together with a plasmid for gene delivery [C. Wang et al., Journal of Controlled Release: Official Journal of the Controlled Release Society, 2012]. However, since iron oxide is a relatively poor T2-type MRI contrast agent for the lung [H. B. Na et al., Angew Chem Int Ed Engl 2007, 46, 5397], there is a need to develop nanoparticles containing T1 contrast agents for better lung imaging that can also be used for drug delivery in lung diseases.

Currently, T1 MRI utilizes predominantly gadolinium- (Gd-)based contrast agents because of the large magnetic moment of Gd³⁺ due to its seven unpaired electrons and slow electronic relaxation time [D. Pan et. al., Wiley Interdisciplinary Reviews: Nanomedicine and Nanobiotechnology, 2011, 3(2), 162; D. Pan et al., Tetrahedron 2011, 67, 8431]. The high toxicity of Gd³⁺, however, requires that these contrast agents always be given in a chelated form. Despite this, several cases of nephrogenic systemic fibrosis (NSF) have been reported in patients receiving Gd-containing contrast agents [M. R. Prince et al., Radiographics: A Review Publication of the Radiological Society of North America, Inc., 2009, 29, 1565; M. A. Sieber et al., Journal of Magnetic Resonance Imaging: JMRI 2009, 30, 1268]. Hence, alternatives to Gd-containing T1 contrast agents are needed.

### BRIEF DESCRIPTION OF THE DRAWINGS

FIG. 1 shows a schematic representation of multi-functional lipid-micellar nanoparticles. PL-1=payload 1; PL-2=payload 2.

FIG. 2 (A-C) shows cell uptake, viability, and in vivo biodistribution of M-LMNs. (A) HEK293 cells were incubated with M-LMNs (10 μg/ml) for 4 hours and cell uptake was determined by laser scanning confocal microscopy; z-stacked images of HEK293 cells showing uptake of rhodamine-conjugated M-LMNs. 1000× magnification is shown. (B) Effects of M-LMN exposure in HEK293 cells. HEK293 cells were incubated for 72 hours with various concentrations of M-LMNs and cell viability was assessed. (C) In vivo biodistribution of Cy5.5-M-LMNs. Groups of mice (n=4) were injected intravenously (IV) or intranasally (IN) with Cy5.5 M-LMNs and at 24, 48 and 96 hours after administration, the Cy 5.5 levels were quantitated by Xenogen imaging. Control mice received PBS alone (IN). Relative fluorescent intensity per mg tissue is shown.

FIG. 3 (A-B) shows (A) Gel electrophoresis (1% Agarose) of the complexes of M-LMNs and DNA at different LMN:DNA weight ratios. (B) Gel electrophoresis (0.8% Agarose) of complexes of M-LMNs and DNA after exposure to DNase I.

FIG. 4 (A-D) shows the gene delivery potential of M-LMNs. HEK293 cells were transfected with M-LMNs complexed with ptd DNA encoding red-fluorescent protein (RFP). Transfection efficiency was monitored by fluorescent microscopy. RFP images (upper panel) and merge of fluorescent images (20× magnification) and phase-contrast (bottom panel) are shown. (A-B) Various ratios of M-LMN:DNA (wt/wt) were incubated with HEK293 cells for 48 hours. The transfected cells were counted with imageJ and the percent transfection in groups was compared with Graphpad Prism. 5:1 vs 10:1 *p<0.05. (C-D) Nanocomplexes of M-LMN:DNA wt/wt; 5:1 were incubated for the indicated length of time. The transfected cells were marked and counted using ImageJ software. The groups were statistically compared in Graphpad Prism. 24 hours versus 48 hours **p<0.01; 48 hours versus 96 hours ***p<0.001.

FIG. 5 (A-D) shows the MRI potential of M-LMNs containing different concentrations of Mn²⁺. Two hundred μl aliquots of indicated concentrations of M-LMNs were added to a 96-well plate in duplicate. T1 relaxometry map derived from the multi-TE T1 measurements, (A) Visual and (B) quantitative T1 MRI contrast is shown. 50 μl of a 0.7 mM Mn solution of M-LMNs were injected intranasally to mice. After one hour the lungs were collected and imaged ex vivo using MRI. (C) Visual and (D) quantitative T1 MRI contrast are shown.

FIG. 6 (A-F) shows the cellular uptake, viability and in vivo biodistribution of D-LMNs. (A) TEM of D-LMNs; scale bar=100 nm. (B) Laser scanning confocal microscopic images (1000× magnification) (z-stacked) of uptake of D-LMNs by HEK293 cell. (C) Release of DOX from D-MLNs in PBS at pH 7.3 and pH 5.4 as a percentage of total encapsulated DOX. Free DOX was used as control. (D) Effect of D-LMNs on viability of LLC1 cells. Cells were incubated for 72 hours with various concentrations of M-LMNs or D-LMNs, and viability was assessed by Presto Blue assay. (E) Comparison of exposure of D-LMNs with free DOX in LLC1 cells. (F) In vivo bio-distribution of D-LMNs. Groups of mice (n=4) were treated intranasally with six rounds of D-LMNs over a two-week period, the DOX levels in each organ were quantitated by Xenogen imaging. Control mice received PBS. Relative fluorescent intensity per mg tissue is shown.

FIG. 7 (A-F) shows cellular uptake, viability, gene transduction, and imaging potential of DM-LMNs. (A) Laser scanning confocal microscopic images (630× magnification; z-stacked) of HEK293 cells showing uptake of D-LMNs. (B) Treatment of LLC1 cells with D-LMNs compared to free DOX. LLC1 cells were incubated for 72 hours with various concentrations of D-LMNs or free DOX and cell viability was determined. (C-D) Transfection potential of DM-LMNs. HEK293 cells were transfected with DM-LMNs complexed with ptdTomato plasmid DNA at wt/wt ratios of 5:1 or 10:1. Transfection efficiency was determined by fluorescence microscopy. Red fluorescent protein (upper panel) and the merge of RFP and the phase-contrast image (bottom panel) (200× magnification) are shown. Transfected cells were counted separately using ImageJ software. The percent of transfected cells were compared with Graphpad Prism. *p<0.05. (E) Simultaneous green fluorescent protein transfection and DOX delivery by DM-LMNs in HEK293 cells. (F) In vivo EGFP-DNA transfection by M-LMNs (a-b) and simultaneous EGFP-DNA transfection and DOX delivery by DM-LMNs (c-e) in mouse lungs (1000× magnification) after 72 hours.

### DETAILED DESCRIPTION

Provided herein is a micelle composition comprising a polyethylene glycol (PEG), a DC-cholesterol, and a dioleoylphosphatidyl-ethanolamine (DOPE) and either or both a pharmaceutical compound core and a polynucleotide coating. Also provided herein is a method of administering one or more compounds to a cell comprising, contacting the cell with a micelle composition comprising 1) a polyethylene glycol-phosphatidyl ethanolamine (PEG-PE), a DC-cholesterol, and a dioleoylphosphatidyl-ethanolamine (DOPE), and 2) the one or more compounds, wherein the compounds are selected from the group consisting of a polynucleotide and a pharmaceutical composition. Further provided are methods for detecting the micelle composition. Term definitions used in the specification and claims are as follows.

Definitions

As used in the specification and claims, the singular form “a,” “an,” and “the” include plural references unless the context clearly dictates otherwise. For example, the term “a cell” includes a plurality of cells, including mixtures thereof.

The term “active derivative” and the like means a modified PEG-PE, DC-cholesterol, or DOPE composition that retains an ability to form a micelle that protects a polynucleotide from nuclease digestion. Assays for testing the ability of an active derivative to perform in this fashion are known to those of ordinary skill in the art.

When referring to a subject or patient, the term “administering” refers to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-peritoneal, intra-synovial, intrasternal, intrathecal, intrahepatic, intralesional, and intracranial injections or infusion techniques. Is some embodiments, the administration is intranasal.

The term “antibody” is used in the broadest sense and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules that lack target specificity. Native antibodies and immunoglobulins are usually heterotetrameric glycoproteins of about 150,000 Daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end. An antibody “specific for” another substance binds, is bound by, or forms a complex with that substance.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such a molecule from having the ability to bind to the high affinity receptor, FcεRI. As used herein, “functional fragment” with respect to antibodies refers to Fv, F(ab) and F(ab′)₂ fragments. The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which enables the sFv to form the desired structure for target binding.

As used herein, the terms “cancer,” “cancer cells,” “neoplastic cells,” “neoplasia,” “tumor,” and “tumor cells” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures. The cancer can be selected from astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is prostate cancer.

It should be understood that the term “coating” describes the method of applying a compound such as a polynucleotide to a pre-formed micelle and does not necessarily indicate the ultimate location of the compound on the exterior of the micelle. It should be further understood that the term “coating” does not require a complete coverage of the coated object and that partial coverage is encompassed by the term.

As used herein, the term “comprising” is intended to mean that the compositions and methods include the recited elements, but not excluding others. “Consisting essentially of,” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

An “effective amount” is an amount sufficient to effect beneficial or desired results. An effective amount can be administered in one or more administrations, applications or dosages.

As used herein, “expression” refers to the process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell. “Overexpression” as applied to a gene refers to the overproduction of the mRNA transcribed from the gene or the protein product encoded by the gene, at a level that is 2.5 times higher, preferably 5 times higher, more preferably 10 times higher, than the expression level detected in a control sample.

A “gene” refers to a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” refers to the amino acid (e.g., peptide or polypeptide) generated when a gene is transcribed and translated.

“Humanized” forms of non-human (e.g. murine) antibodies are chimeric immunoglobulins, immunoglobulin chains or fragments thereof (such as Fv, Fab, Fab′, F(ab′)₂ or other target-binding subsequences of antibodies) that contain minimal sequence derived from non-human immunoglobulin. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody may also comprise at least a portion of an immunoglobulin constant region (Fc), typically that of a human immunoglobulin template chosen.

The term “isolated” means separated from constituents, cellular and otherwise, in which the polynucleotide, peptide, polypeptide, protein, antibody, or fragments thereof are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof does not require “isolation” to distinguish it from its naturally occurring counterpart. In addition, a “concentrated,” “separated,” or “diluted” polynucleotide, peptide, polypeptide, protein, antibody, or fragments thereof is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

As used herein, the term “micelle” refers to a single layer aggregation of molecules wherein hydrophobic portions of the molecules comprise the interior of the aggregation and hydrophilic portions of the molecules comprise the exterior of the aggregation. Accordingly, the term “micelle” refers herein to the molecules that aggregate to form the micelle, for example, polyethylene glycol-phosphatidyl ethanolamine (PEG-PE), 3β-[N-(N′,N′-dimethylaminoethane)-carbamoyl] cholesterol (DC-cholesterol), and dioleoylphosphatidyl-ethanolamine (DOPE). The term “single layer” excludes bilayer compositions such as liposomes from the definition of a micelle. Liposomes are structurally different from micelles in that they have a bilayer membrane. This bilayer nature provides benefits for drug delivery that are not found in single layer aggregations such as micelles. Further, when a compound resides in a “micelle core,” that compound resides in the interior of the micelle aggregation. When a compound is coated onto the exterior of a micelle, that compound can ultimately reside in the exterior of the micelle aggregation, reside in the interior of the micelle aggregation, or reside in both the exterior and interior portions of the micelle aggregation. As described herein, “micelle compositions” contain materials in addition to the micelle itself, such as core compounds and coated compounds.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies for use with the present invention may be isolated from phage antibody libraries using well-known techniques. The parent monoclonal antibodies to be used in accordance with the present invention may be made by the hybridoma method or may be made by recombinant methods.

A “pharmaceutical composition” is intended to include the combination of an active agent with a carrier, inert or active, making the composition suitable for diagnostic or therapeutic use in vitro, in vivo or ex vivo.

The term “pharmaceutically acceptable carrier or excipient” means a carrier or excipient that is useful in preparing a pharmaceutical composition that is generally safe, non-toxic and neither biologically nor otherwise undesirable and includes a carrier or excipient that is acceptable for veterinary use as well as human pharmaceutical use. A “pharmaceutically acceptable carrier or excipient” as used in the specification and claims includes both one and more than one such carrier or excipient. As used herein, the term “pharmaceutically acceptable carrier” encompasses any of the standard pharmaceutical carriers, such as a phosphate buffered saline solution, water, emulsions, such as an oil/water or water/oil emulsion, and various types of wetting agents. The compositions also can include stabilizers and preservatives.

The term “pharmaceutically acceptable salts” refers to any acid or base addition salt whose counter-ions are non-toxic to the subject to which they are administered in pharmaceutical doses of the salts. Specific examples of pharmaceutically acceptable salts are provided below.

The terms “pharmaceutically effective amount,” “therapeutically effective amount,” or “therapeutically effective dose” refer to the amount of a compound that will elicit the biological or medical response of a tissue, system, animal, or human that is being sought by the researcher, veterinarian, medical doctor or other clinician.

The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disorder or conditions and their severity, the route of administration, time of administration, rate of excretion, drug combination, judgment of the treating physician, dosage form, and the age, weight, general health, sex and/or diet of the subject to be treated.

The terms “polynucleotide” and “oligonucleotide” are used interchangeably and refer to a polymeric form of nucleotides of any length, either deoxyribonucleotides or ribonucleotides, or analogs thereof. Polynucleotides may have any three-dimensional structure and may perform any function, known or unknown. The following are non-limiting examples of polynucleotides: a gene or gene fragment, exons, introns, messenger RNA (mRNA), transfer RNA, ribosomal RNA, siRNA, ribozymes, cDNA, recombinant polynucleotides, branched polynucleotides, plasmids, vectors, isolated DNA of any sequence, isolated RNA of any sequence, polynucleotide probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine (T) when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Selectively binds” refers to a non-specific binding event as determined by an appropriate comparative control. Binding is selective when the binding is at least 10, 30, or 40 times greater than that of background binding in the comparative control.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

“Transformation” of a cellular organism with DNA means introducing DNA into an organism so that the DNA is replicable, either as an extrachromosomal element or by chromosomal integration. “Transfection” of a cellular organism with DNA refers to the taking up of DNA, e.g., an expression vector, by the cell or organism whether or not any coding sequences are in fact expressed. The terms “transfected host cell” and “transformed host cell” refer to a cell in which DNA was introduced. The cell is termed “host cell” and it may be either prokaryotic or eukaryotic. Typical prokaryotic host cells include various strains of E. coli. Typical eukaryotic host cells are mammalian, such as Chinese hamster ovary cells or cells of human origin. The introduced DNA sequence may be from the same species as the host cell of a different species from the host cell, or it may be a hybrid DNA sequence, containing some foreign and some homologous DNA.

The term “vector” means a DNA construct containing a DNA sequence which is operably linked to a suitable control sequence capable of effecting the expression of the DNA in a suitable host. Such control sequences include a promoter to effect transcription, an optional operator sequence to control such transcription, a sequence encoding suitable mRNA ribosome binding sites, and sequences which control the termination of transcription and translation. The vector may be a plasmid, a phage particle, or simply a potential genomic insert. Once transformed into a suitable host, the vector may replicate and function independently of the host genome or may, in some instances, integrate into the genome itself. In the present specification, “plasmid” and “vector” are sometimes used interchangeably, as the plasmid is the most commonly used form of vector. However, the invention is intended to include such other forms of vectors which serve equivalent function as and which are, or become, known in the art.

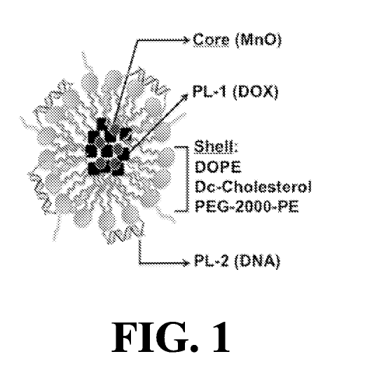

Accordingly, provided herein is a micelle composition comprising a polyethylene glycol-phosphatidyl ethanolamine (PEG-PE), a 3β-[N-(N′,N′-dimethylaminoethane)-carbamoyl] cholesterol (DC-cholesterol), and a dioleoylphosphatidyl-ethanolamine (DOPE) and either or both a pharmaceutical compound core and a polynucleotide coating. In some embodiments, the micelle composition further comprises an imaging contrast agent. These cationic lipid micellar nanoparticles are referred to herein as “LMNs.” FIG. 1 provides a general schematic of the micelle composition.

The polyethylene glycol-phosphatidyl ethanolamine (PEG-PE) found in the micelle composition can be of any molecular weight that allows for formation of a micelle with DC-cholesterol and DOPE, The PEG-PE compound includes PEG molecules having an average molecular weight between approximately 570-630 Da (PEG 600), 720-880 Da (PEG 800), 950-1050 Da (PEG 1000), 1800-2200 Da (PEG 2000), 2700-3300 Da (PEG 3000), 3500-4500 Da (PEG 4000), or 5000-7000 Da (PEG 6000). In one embodiment, the PEG-PE compound comprises PEG molecules having an average molecular weight between approximately 1800-2200 Da. Accordingly, included in the present invention is a micelle composition comprising 1,2-dioleoyl-sn-glycero-3-phosphoethanolamine-N-[methoxy(polyethylene glycol)-2000]. It should be understood that most PEG compounds include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). MW and Mn can be measured by mass spectrometry.

It should be understood that the PEG-PE, DC-cholesterol, and DOPE can be present in any ratio or percentage that allows for micelle formation. In some embodiments, the micelle comprises between approximately 1-10%, 50-75%, and 15-40% of the PEG-PE, the DC-cholesterol, and the DOPE, respectively. Accordingly, the micelle composition can comprise approximately 1%, 2%, 3%, 4%, 5%, 6%, 7%, 8%. 9%, or 10% of a PEG-PE. The micelle composition can comprise approximately 50%, 55%, 60%, 65%, 70%, or 75% of a DC-cholesterol. The micelle composition can comprise approximately 15%, 20%, 25%, 30%, 35%, or 40% of a DOPE. In one embodiment, the micelle comprises between approximately 2%, 66%, and 32% of a PEG-PE, a DC-cholesterol, and a DOPE, respectively.

The micelle compositions provided herein comprise either or both a pharmaceutical compound core and a polynucleotide coating. It should be understood that the pharmaceutical can be any compound that is hydrophobic or that can be made to be hydrophobic. In one embodiment, the pharmaceutical is a cancer chemotherapy compound, and in certain further embodiments, the pharmaceutical is doxorubicin. A micelle comprises a pharmaceutical compound core when the pharmaceutical resides in the core, or interior, of the micelle.

Micelle compositions that comprise a “polynucleotide coating” refer to pre-formed micelle compositions to which polynucleotides are applied. When a polynucleotide is coated onto the exterior of a micelle, that polynucleotide can ultimately reside in the exterior of the micelle aggregation, reside in the interior of the micelle aggregation, or reside in both the exterior and interior portions of the micelle aggregation. The polynucleotides can be of any length, either deoxyribonucleotides or ribonucleotides, or analogs thereof. Polynucleotides may have any three-dimensional structure and may perform any function, known or unknown. The following are non-limiting examples of polynucleotides: a gene or gene fragment, exons, introns, messenger RNA (mRNA), transfer RNA, ribosomal RNA, siRNA, ribozymes, cDNA, recombinant polynucleotides, branched polynucleotides, plasmids, vectors, isolated DNA of any sequence, isolated RNA of any sequence, polynucleotide probes, and primers. The micelle compositions provided herein can comprise any amount of polynucleotide. In some embodiments, the polynucleotide coating is applied to a micelle at a micelle:polynucleotide molecular weight ratio of approximately 15:1, 10:1, 5:1, 2:1, or 1:1.

In some embodiments, the micelle composition further comprises a hydrophobic contrast imaging agent located in its core. Contrast imaging agents include, but are not limited to, T1 magnetic resonance imaging (MRI) agents and T2 MRI agents. In one embodiment, the contrast imaging agent is a T1 MRI agent. T1 MRI agents include manganese (Mn), and the present invention includes micelles having a hydrophobic manganese core. In some embodiments, the micelle composition comprises a manganese-oleate core.

The nonlanthanide metal manganese (Mn) is paramagnetic, has five unpaired electrons in its bivalent state, and is a natural cellular constituent as a cofactor for enzymes and receptors [D. Pan et al., Wiley Interdisciplinary Reviews: Nanomedicine and Nanobiotechnology, 2011, 3(2), 162; D. Pan et al., Tetrahedron 2011, 67, 8431]. The intrinsic properties of Mn include high spin number, long electronic relaxation time, and labile water exchange. Though Mn-containing contrast agents are FDA approved for clinical use [D. Pan et al., Wiley Interdisciplinary Reviews: Nanomedicine and Nanobiotechnology, 2011, 3(2), 162], Mn can be toxic at the high levels required to offset the short plasma half-life of ionic Mn [D. Pan et al., Tetrahedron 2011, 67, 8431; J. Y. Choi et al., Bioprocess and Biosystems Engineering 2010, 33 21]. In formulating the micelles described herein, it was hypothesized that sequestration of Mn within nanoparticles could possibly reduce the risk of toxicity and overcome the problem of short plasma half-life.

Accordingly, provided herein are micelle compositions comprising a PEG-PE, a DC-cholesterol, and a DOPE and further comprising a pharmaceutical compound core, a hydrophobic imaging agent core, and/or a polynucleotide coating. In one embodiment, the micelle composition comprises a PEG-PE, a DC-cholesterol, and a DOPE and further comprises a pharmaceutical compound core. In another embodiment, the micelle composition comprises a PEG-PE, a DC-cholesterol, and a DOPE and further comprises a pharmaceutical compound core and a polynucleotide coating. In yet another embodiment, the micelle composition comprises a PEG-PE, a DC-cholesterol, and a DOPE and further comprises a pharmaceutical compound and hydrophobic imaging agent core and a polynucleotide coating. In a still further embodiment, the micelle composition comprises a PEG-PE, a DC-cholesterol, and a DOPE and further comprises a polynucleotide coating and a hydrophobic imaging agent core.

In some embodiments, the micelle composition further comprises a ligand. A ligand is defined herein as any moiety that facilitates binding of the compositions provided herein to a target such as a cell. Ligands include, but are not limited to, antibodies, adhesion molecules, lectins, integrins, and selectins. When the ligand is an antibody, it can comprise approximately 1% of the total composition weight (but is not limited to such amount). In some embodiments, the ligand is an antibody specific for a cancer cell.

The micelle compositions provided herein are useful for administering polynucleotides, pharmaceutical compositions, and/or MRI imaging agents to cells, and in particular, to cells in a subject. The examples below describe in vitro MRI, cellular uptake, transfection, cytotoxicity studies, and in vivo experiments in mice which demonstrate that these cationic lipid nanoparticles act as a T1 contrast agent and DNA/drug delivery vehicle. It was a surprising finding of the present invention that the unique combination of DOPE, DC-cholesterol and PEG-2000-PE yielded a high gene transfection efficiency and drug uptake. When administered to mice intranasally as nasal drops, the DM-LMN nanoparticles were found mostly in the lungs, in marked contrast to other polymers, making them an ideal candidate for lung cancer theranostics.

Accordingly, provided herein is a method of administering one or more compounds to a cell comprising, administering to the cell a micelle composition comprising 1) a PEG-PE, a DC-cholesterol, and a DOPE, and 2) the one or more compounds, wherein the compounds are selected from the group consisting of a polynucleotide and a pharmaceutical composition. In some embodiments, the administered micelle composition comprises a polynucleotide coating as described above. When administering a micelle composition comprising a polynucleotide, the method can include transfecting and/or transforming a cell to which the micelle composition is administered. In other embodiments, the administered micelle composition comprises a pharmaceutical compound core as described above. In still other embodiments, the administered micelle composition comprises both a polynucleotide coating and a pharmaceutical compound core.

The micelle composition can be administered to a cell in vitro, in vivo, or ex vivo. In one embodiment, the micelle composition is administered to a subject. When referring to a subject or patient, the terms “administered” and “administering” refer to an administration that is oral, topical, intravenous, subcutaneous, transcutaneous, transdermal, intramuscular, intra joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-peritoneal, intra-synovial, intrasternal, intrathecal, intrahepatic, intralesional, and intracranial injections or infusion techniques. The methods can also comprise placing a magnet proximal to a target cell or group of target cells prior to, during, and/or after administration of the micelle composition to a subject containing the cell(s). A target cell is that cell to which delivery of the micelle composition is desired. In some embodiments, the target cells are lung cells.

The examples below indicate that intranasal administration of the micelle composition provided herein to a subject directs the micelle composition to the lungs of the subject. Accordingly, in some embodiments, the administration is intranasal. In still further embodiments, the administration is intranasal and the cell to which the micelle composition is delivered is a lung cell.

The examples below further indicate that the micelle composition described herein can contain an MRI imaging agent, which agent permits the visualization of the micelle composition after it is administered to a subject. Accordingly, included herein is a method of administering one or more compounds to a subject comprising, administering to the subject a micelle composition comprising 1) a PEG-PE, a DC-cholesterol, and a DOPE, 2) the one or more compounds, wherein the compounds are selected from the group consisting of a polynucleotide and a pharmaceutical composition, and 3) an MRI imaging agent. In one embodiment, the MRI imaging agent is a hydrophobic manganese-oleate core. Also provided herein is a method of detecting the administration of one or more compounds to a subject comprising, administering to the subject a micelle composition comprising 1) a PEG-PE, a DC-cholesterol, and a DOPE, and 2) the one or more compounds, wherein the compounds are selected from the group consisting of a polynucleotide and a pharmaceutical composition, and 3) an MRI imaging agent; and detecting a location of the micelle composition in the subject using magnetic resonance imaging technology. In one embodiment, the administration of a polynucleotide is detected. In another embodiment, the administration of a pharmaceutical compound is detected.

It should be understood that the foregoing relates to preferred embodiments of the present disclosure and that numerous changes may be made therein without departing from the scope of the disclosure. The disclosure is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present disclosure and/or the scope of the appended claims. All patents, patent applications, and publications referenced herein are incorporated by reference in their entirety for all purposes.

### EXAMPLES

### Example 1

### Preparation and Characterization of Manganese LMNs (M-LMNs)

To prepare M-LMNs, Mn²⁺-oleate complexes were subjected to thermal decomposition in a high boiling-point solvent that produces MnO with a hydrophobic surface layer of oleic acid. To create a hydrophilic exterior, phospholipid micelles encapsulating these MnO nanoparticles were prepared by the thin-film hydration method in which hydrophobic MnO nanoparticles were added to a mixture of PEG-2000 PE, DC-cholesterol and DOPE dissolved in chloroform. The particles were vacuum dried and the dry film was swelled in water, sonicated and centrifuged to remove uncoated MnO nanoparticles. The micelles coating the MnO nanoparticles are composed of ingredients that have been FDA approved for use in humans or have been used in clinical trials. The lipids DOPE and DC-cholesterol have been used in clinical trials for the nasal delivery of DNA to cystic fibrosis patients [D. R. Gill et al., Gene Therapy 1997, 4, 199; N. J. Caplen et al., Nature Medicine 1995, 1, 39; S. C. Hyde et al., Gene Therapy 2000, 7, 1156; P. G. Middleton et al., The European Respiratory Journal: Official Journal of the European Society for Clinical Respiratory Physiology 1994, 7, 442]. Lipid-conjugated PEG-2000 is an essential part of the FDA-approved formulation Doxil® [Y. Barenholz, Journal of Controlled Release: Official Journal of the Controlled Release Society 2012, 160, 117].

More specifically, the following materials and methods were used.

Materials: Manganese sulfate, sodium oleate, chloroform, hexane, 1-octadecane, dichloromethane, triethylamine, and acetone were all purchased from Sigma. PEG-2000 PE, DC-cholesterol, DOPE, and 1,2-dipalmitoyl-sn-glycero-3-phosphoethanolamine-N-lissamine rhodamine B sulfonyl were all purchased from Avanti Polar Lipids. Cy5.5 NHS was purchased from Invitrogen. All reagents were used without further purification.

Synthesis of Mn-oleate complexes: Mn-oleate was prepared by the method described previously with some modifications [J. Park et al., Nature Materials 2004, 3, 891]. Manganese sulfate (2 g) and sodium oleate (6.1 g) were dissolved in a combination of ethanol (7.5 ml), hexane (17 ml), and distilled water (10 ml). The solution was heated to 70° C. with vigorous stirring overnight. The solution was then transferred to a separatory funnel and the upper organic layer containing the Mn-oleate complex was washed several times using distilled water. The purified solution was allowed to evaporate, producing a deep red waxy solid that was the manganese-oleate complex.

Hydrophobic MnO nanoparticles were then prepared by the method described previously with some modifications [J. Park et al., Nature Materials 2004, 3, 891]. Manganese-oleate (1.3 g) was dissolved in 1-octadecene (13.5 ml), and degassed at 70° C. for 1 hour under vacuum with vigorous stirring. The solution was then purged with argon and heated to 300° C. while stirring under argon. As the temperature reached 300° C., the initially red solution turned transparent and then pale green. The reaction was held at this temperature for 1 hour and 15 minutes, then allowed to cool to room temperature after which dichloromethane (20 ml) was added to improve the dispersibility of the nanoparticles. Acetone (80 ml) was added to precipitate the nanoparticles and the solution was centrifuged at 3,000 rpm (835×g) at 4° C. for 15 minutes. Supernatants were discarded and the pellets were reconstituted in 20 ml of dichloromethane. The above purification procedure was repeated two more times to remove excess surfactant and solvent. The purified MnO nanoparticles were dispersible in many organic solvents such as dichloromethane and chloroform.

Preparation of MnO nanoparticles encapsulated in micelles: MnO nanoparticles were encapsulated inside micelles using a published procedure with some modifications [R. Kumar et al., Theranostics 2012, 714-722; Y. Namiki et al., Nature Nanotechnology 2009, 4, 598; B. Dubertret et al., Science 2002, 298, 1759]. PEG-2000 PE (0.1 mg, 2% of total), DC-cholesterol (7.9 μM, 3.95 mg, 66% of total), and DOPE (2.6 μM, 1.95 mg, 32% of total) were added to 1.5 ml of chloroform. Then 3 mg (0.23 ml of stock solution) of MnO nanoparticles were added to this solution. To ensure complete solubilization, the reaction solution was sonicated in a Branson 2510 sonicator for 20 minutes. The solution was then left to evaporate overnight in a vacuum oven at 40° C. The dry film was heated at 80° C. for 2 minutes. Then 2 ml of water was added and the solution was again sonicated for 3 hours. After the film was dissolved, the solution was centrifuged at 90,000 rpm (334,000×g) at 4° C. for 2 hours to separate filled micelles from empty ones. The pellet was reconstituted in 1 ml of water and sonicated further for 30 minutes. The M-LMNs were filtered through a 0.45-micron syringe filter and stored at 4° C.

Chemical and physical characterization of nanoparticles: FTIR spectra of oleic acid-coated MnO nanoparticles were obtained using a Nicolet IR-100 spectrometer. A 20 μl aliquot of the oleic acid-coated MnO nanoparticles dispersed in chloroform was dropped onto a disposable polyethylene IR card and the solvent was evaporated under vacuum before taking the measurements. FTIR spectrometry was performed on the MnO-oleate nanoparticles and bands characteristic of oleic acid-coated hydrophobic MnO nanoparticles were identified. The surface-bonded oleic acid was confirmed by the presence of bands in the 2900 and 2850 cm⁻¹ range, due to the C—H stretch, and a band at 1461 cm⁻¹ (C—H bending) [C. Wang et al., Journal of Controlled Release: Official Journal of the Controlled Release Society, 2012].

The morphology and size of nanoparticles were determined using transmission electron micrograph (TEM) and dynamic light scattering (DLS). TEM of MnO nanoparticles in chloroform and aqueous M-LMNs was performed by pipetting 10 μl of diluted stock solution (0.25 mg/ml) onto a carbon-coated copper grid. The MnO grid was allowed to air-dry for one hour before visualization and the M-LMNs grid was allowed to air-dry overnight. Once dry the M-LMNs grid was then negatively stained using a 1% uranyl acetate solution. The sample was visualized with a JEOL 1200 EX transmission electron microscope at 80 kV. DLS of M-LMNs in aqueous solution was performed using a DynaPro DLS plate reader. To prepare DLS samples, the M-LMNs stock solution was diluted to a concentration of 0.25 mg/ml and sonicated for 30 minutes to prevent aggregation. Zeta potential was determined using a MicroTracZetaTrac instrument. To prepare the samples, the M-LMNs stock solution was diluted to 0.25 mg/ml and sonicated for 30 minutes to prevent aggregation.

TEM images of oleate-MnO showed mostly spherical nanoparticles with a size of 10-30 nm. DLS analysis showed the hydrodynamic radius for the M-LMNs to be about 100 nm, which was confirmed by TEM images of M-LMNs where several electron-dense MnO nanoparticles can be seen clustered within an M-LMN particle. The surface charge of these micelles was determined by measuring their zeta potential. M-LMNs showed a net positive zeta potential of +37 mV, most likely due to the cationic DC-cholesterol and DOPE with its primary amine head groups. Inductively-coupled plasma-mass spectrometry was used to determine the concentration of Mn encapsulated in the M-LMNs. The MnO loading efficiency was determined to be about 10%.

### Example 2

### Cellular Uptake, Cytotoxicity and in vivo Biodistribution of M-LMNs

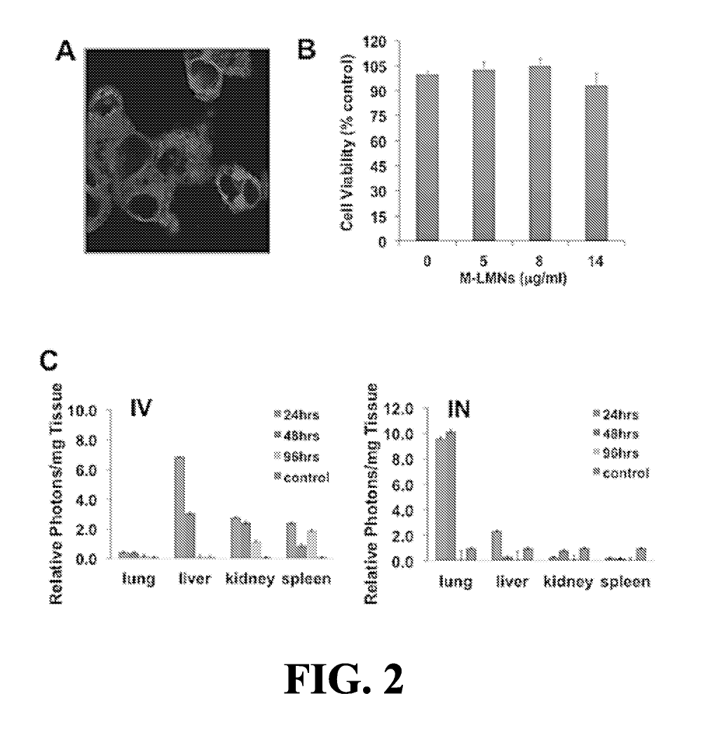

To examine the cellular uptake of M-LMNs, the particles were labeled with the fluorescent lipid, 1,2-dipalmitoyl-sn-glycero-3-phosphoethanolamine-N-lissamine rhodamine B sulfonyl. More specifically, fluorescent M-LMNs (FM-LMNs) were prepared as previously described with some modifications. The fluorescent-labeled lipid 1,2-dipalmitoyl-sn-glycero-3-phosphoethanolamine-N-lissamine rhodamine B sulfonyl (0.2 mg) was added to the initial lipid mixture in chloroform during micelle preparation. For the uptake experiments, cells were seeded 24 hours prior to transfection into an 8-well chamber slide at a density of 80,000 cells per well in 300 μl of complete medium (DMEM containing 10% FBS, 2 mM L-glutamate and 1% penicillin/streptomycin). At the time of FM-LMNs addition, the medium in each well was replaced with 250 μl of fresh DMEM with no FBS. Various amounts of FM-LMNs, diluted in 50 μl DMEM with no FBS, were added to each well. After 4 hours of incubation, the cells were washed with PBS and fixed to the slide using a 10% neutral buffered formalin solution. Nuclei of the cells were stained using DAPI. The distribution of FM-LMNs inside the cells was imaged with the multiphoton Olympus BX61W1 confocal microscope. Human embryonic kidney HEK293 cells were incubated with labeled M-LMNs for 4 hours and the dye was visualized by confocal microscopy (FIG. 2A). M-LMNs were seen in the cytoplasm surrounding the nuclei of the cells.

To determine the cytotoxicity of these micelles, cell viability was measured using the PrShinaesto Blue assay. More specifically, in vitro cytotoxicity was evaluated in HEK293 cells and LLC1 cells using the PrestoBlue® assay (Roche) according to the manufacturer's specifications. Cells were seeded 24 hours prior to transfection into a 96-well plate in 100 μl of complete medium (DMEM containing 10% FBS, 2 mM L-glutamate and 1% penicillin/streptomycin). At the time of nanoparticle addition, the medium in each well was replaced with 50 μl of fresh complete DMEM. Various concentrations of the nanoparticles were diluted in 50 μl DMEM with no FBS and added to the well in triplicate. The cells were cultured in an incubator at 37° C. under 5% CO₂ and viability was determined after 72 hours. Cells without nanoparticles were used as a control with viability taken as 100%. FIG. 2B shows that M-LMNs demonstrated no apparent toxicity when incubated with the HEK cells at any of the concentrations tested.

To determine the particles' biodistribution in vivo, M-LMNs were labeled with Cy5.5, a near-infrared imaging dye. The resulting particles were administered intravenously (IV) or intranasally (IN) to groups of C57BL/6 mice. Control mice were administered PBS (IN). Twenty-four, 48 and 96 hours after treatment, lung, liver, kidney, and spleen were excised and examined for Cy5.5 using a Xenogen IVIS imager and quantified. Twenty-four hours after IV administration, the Cy5.5-M-LMNs were found mostly distributed in the liver and also in the kidney and spleen, but not in the lungs of mice (FIG. 2C). In sharp contrast, intranasally administered Cy5.5-M-LMNs were found preferentially (approximately 85% of total) accumulated in the lungs for up to 48 hours (FIG. 2C), demonstrating that M-LMNs have the potential to be used as theranostics for lung disease.

### Example 3

### Gene Delivery Potential of M-LMNs

In order for the micelles to act as a gene delivery vehicle, they must be able to form a stable complex with nucleic acids during transport and entry to release the DNA within the cells. The capability of these cationic lipid micelles to form complexes with DNA was evaluated using a gel-retardation assay. More specifically, M-LMN complexes with different ratios of micelle:DNA were tested. M-LMNs were diluted with PBS to a final concentration of 2 μg/μl and aliquots of M-LMNs and plasmid DNA solution were diluted separately with an appropriate volume of PBS. The plasmid DNA solution was then added slowly to the M-LMNs solution and vortexed for 30 minutes. The M-LMN:DNA complexes were mixed with loading buffer and loaded into individual wells of a 0.9% agarose gel containing ethidium bromide. Gels were electrophoresed at room temperature in Tris/Borate/EDTA buffer at 120V for 20 minutes. DNA bands were visualized using a ChemiDoc TM XRS imaging system.

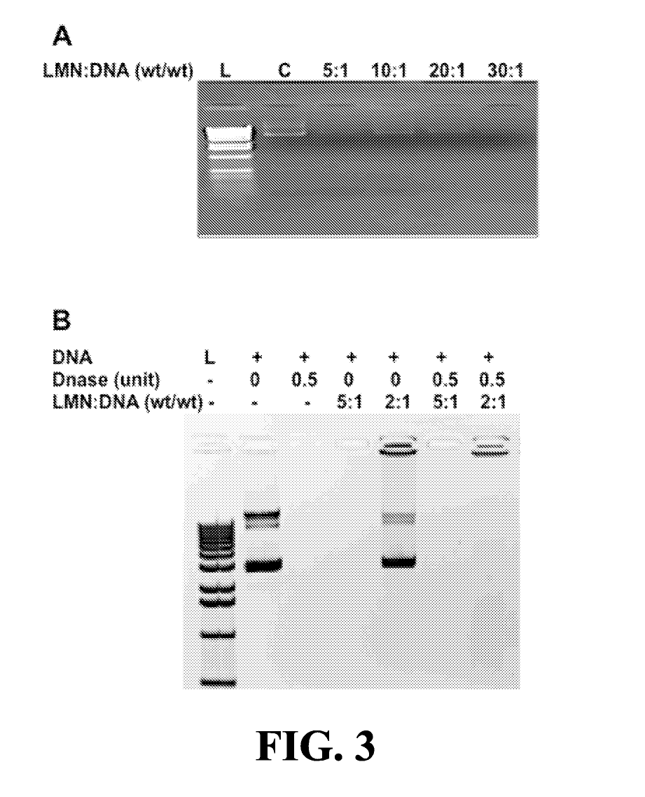

DNA that was bound to the micelles remained in the wells, while unbound DNA migrated down the gel. It was observed that M-LMNs were able to completely retard migration of the DNA at weight ratios as low as 5:1 (M-LMNs:DNA) (FIG. 3). During DNA delivery it is critical to protect the DNA from degradation by nucleases. The absence of ethidium bromide staining in even the wells that contain the M-LMNs:DNA complexes suggest that the M-LMNs are able to fully protect the DNA from the ethidium bromide at weight ratios as low as 5:1 (M-LMNs:DNA).

To further prove that the DNA was protected from nucleases once complexed with the M-LMNs, M-LMNs:ptd-Tomato DNA complexes were exposed to DNase-I (FIG. 3B). More specifically, M-LMN complexes with different micelle:DNA ratios were tested. M-LMNs complexed with 0.5 μg plasmid DNA (pCMV-td-Tomato, Invitrogen) at M-LMN:DNA (wt/wt) 5:1 or 2:1 or 0.5 μg plasmid DNA alone were incubated with 0.5 U DNase I (Roche) for 20 minutes at 37° C. To inactivate the DNase I, the solutions were then incubated at 75° C. for 10 minutes. The samples were then mixed with loading buffer and loaded into individual wells of a 0.8% agarose gel containing ethidium bromide. Gels were electrophoresed at room temperature in Tris/borate/EDTA buffer at 100 V for 40 minutes. DNA bands were visualized using a ChemiDoc TM XRS imaging system.

The presence of partial DNA bands in the lane and ethidium bromide staining in the well of the 2:1 (M-LMNs:DNA wt/wt) complex shown in FIG. 3 suggest that the DNA was only partially complexed to the micelles at this ratio. The DNA that was not fully encapsulated within the micelles was completely degraded, as can be seen from the absence of any DNA bands in this lane. However, the DNA that was fully enclosed in the micelles was protected from DNase I degradation, as can be seen from the ethidium bromide staining present in the well of this lane. At a ratio of 5:1 (M-LMNs: DNA wt/wt) the DNA was fully protected from both DNase I degradation and ethidium bromide staining, as judged by the absence of any bands in the lanes or ethidium bromide staining in these wells. These results suggest that M-LMNs, at a (M-LMNs: DNA wt/wt) ratio of 5:1 or higher are able to completely protect the complexed DNA from nuclease degradation.

The ability of M-LMNs to transduce DNA into cultured cells and achieve protein expression was also determined by using a plasmid DNA encoding red-fluorescent protein (RFP) as a reporter. More specifically, cells were seeded 24 hours prior to transfection into a 96-well plate at a density of 10,000 cells per well in 100 μl of complete medium (DMEM containing 10% FBS, 2 mM L-glutamate and 1% penicillin/streptomycin). At the time of transfection, the medium in each well was replaced with 100 μl of fresh DMEM with no FBS. An amount of M-LMNs equivalent to the desired weight ratio needed for use with 0.2 μg of DNA plasmid expressing red-fluorescent protein (pCMV-td-Tomato, Invitrogen) was added to each well. Four hours after addition of M-LMNs, 50 μl of DMEM containing 40% FBS was added to each well and the plate was incubated for a total of 96 hrs. All images were made with an Olympus IX71 microscope equipped with an EXFO X-Cite Series 120 fluorescence excitation light source (λex=554 nm, λem=581 nm) and a DP-70 high-resolution digital camera. Images were taken at 24, 48, and 96 hours post-transfection.

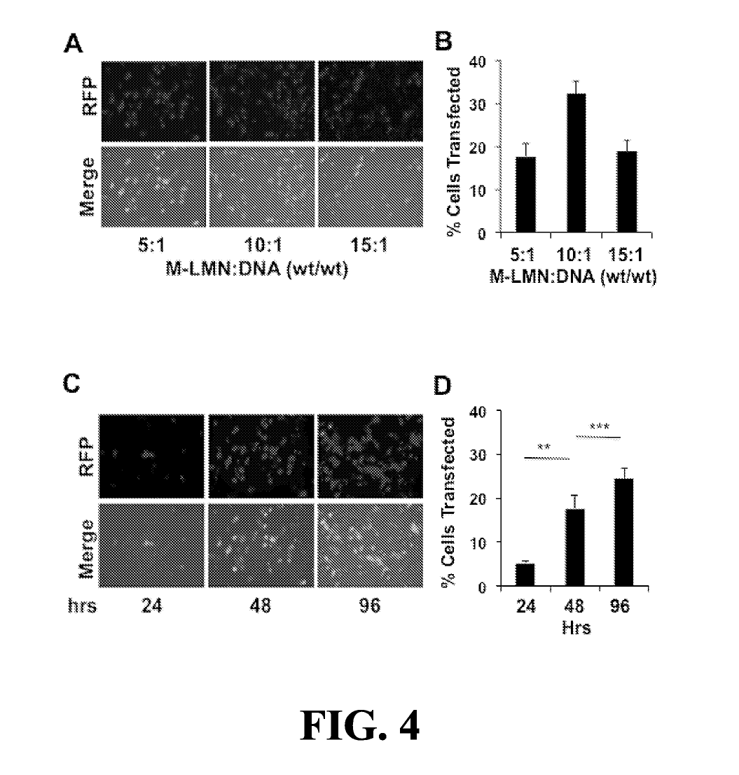

FIG. 4 shows the results of these experiments wherein cells were incubated with various ratios of micelle:pDNA in M-LMN complexes (FIG. 4A and 4B) and for various times (FIG. 4C and 4D). The fluorescent images in FIGS. 4A and 4B show that M-LMNs at micelle:pDNA weight ratios of 5:1 or 10:1 readily transfected HEK293 cells. Transfection was less efficient at a ratio of 15:1. Expression of RFP was seen as early as 24 hours and was maximal at 96 hours (FIG. 4C and 4D). Also, M-LMN:ptd-Tomato DNA (5:1) induced protein expression levels similar to lipofectamine-transfected DNA (data not shown). These results indicate that M-LMNs may be a useful tool for the delivery of DNA into mammalian cells.

### Example 4

### M-LMNs provide MRI Capability in vitro and ex vivo

In addition to administering nucleic acids and small molecules to target sites, this cationic lipid nanoparticle system was also designed to act as a T1 MRI contrast agent to allow monitoring of the effects of gene or drug delivery, It was recognized that phospholipid-encapsulated oleic acid coated nanoparticles are strongly protected from the outside aqueous environment by a tight hydrophobic layer and that this may prevent water protons from contacting the manganese nanoparticle surface and could therefore lead to a lowering of the relaxivity [H. Duan et al., The Journal of Physical Chemistry Letters 2008, 8127]. However, the DOPE component of the MLNs phospholipid micelle has two unsaturated fatty acid tails, which serve to increase the fluidity of the phospholipid micellar membrane. Since T1 contrast agents need to have direct interaction with the surrounding water protons to affect their relaxation times [Z. Zhen & J. Xie, Theranostics 2012, 2, 45], this increased fluidity could allow for more interaction of the manganese oxide nanoparticles and water protons.

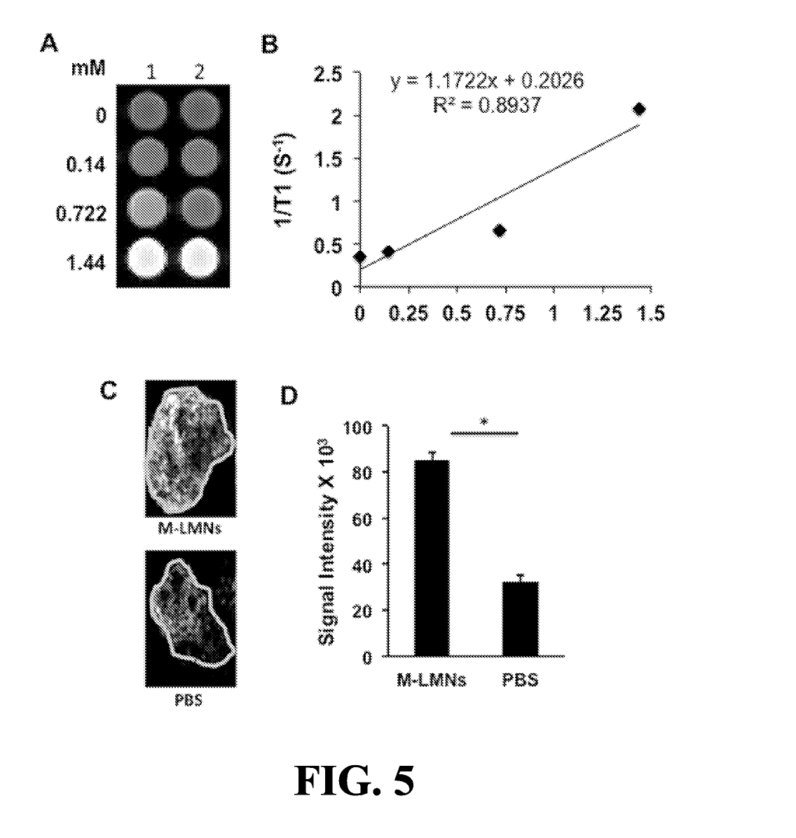

To determine whether M-LMNs were able to act as an effective T1 MRI contrast agent, their relaxation properties were analyzed by MR phantom imaging. Phantom MRI was performed as follows: Various dilutions of M-LMNs and DM-LMNs were diluted in deionized water and the concentration of manganese in the micelles was determined by ICP-MS. Two hundred μl aliquots of the various micelle solutions were added to a 96-well plate in duplicate and MR images were obtained using an Agilent ASR 310 7 Tesla MRI high-field scanner. Fast Spin-Echo Multi-Slice (FSEMS) experiments were performed in imaging mode to determine the measure of T1 values. Nonlinear least-square fitting was performed using the MATLAB program (Mathworks, Inc.) on a pixel-by-pixel basis. A region of interest was drawn for each well, where the mean value was used to determine the longitudinal molar relaxivity r1. The image was recorded with Vnmrj 3.0. FIG. 5 shows the visual (A) and quantitative (B) T1 MRI contrast provided by M-LMNs for various Mn concentrations. The R1 relaxivity of M-LMNs (1.17 mM-1s-1) was larger than the values reported for MnO-SiO2-PEG/NH₂ nanoparticles (0.47 mM-1s-1) [T. D. Schladt et al, Journal of Materials Chemistry 2012, 9253] and was comparable to the values reported for PEG-phospholipid-encapsulated HMONs (1.417 mM-1s-1) [J. Shin et al., Angew Chem Int Ed Engl 2009, 48, 321].

Since M-LMNs preferentially accumulate in the lungs of mice after intranasal administration, whether M-LMNs would enhance the T1 MRI contrast of the lungs was investigated. More specifically, C57B1/6 mice (n=2) were treated with one intranasal instillation of M-LMNs (50 μl of a 0.7 mM Mn solution). Control mice (n=4) were given PBS. After one hour, the mice were euthanized; the lungs were collected and placed into a medical cassette to be viewed. MR images were obtained using an Agilent ASR 310 7 Tesla MRI high-field scanner. Gradient Echo Multi-Slice (GEMS) experiments (flip angle=45°; TR=175) were performed in imaging mode. Nonlinear least-square fitting was performed using the MATLAB program (Mathworks, Inc) on a pixel-by-pixel basis. A region of interest was drawn around each lung and the mean value of the signal intensity was determined in this area. The image was recorded with Vnmrj 3.0. FIG. 5 shows the visual (C) and quantitative (D) T1 MRI contrast provided by M-LMNs in mouse lungs. The calculated mean signal intensity for M-LMN-injected lungs was more than 2.5 times higher than that of the PBS-injected lungs. These results demonstrate that in addition to administering a therapeutic agent, the M-LMNs could potentially act as a T1 MRI contrast agent to enhance detection and provide a more accurate diagnosis or post-therapy evaluation.

### Example 5

### Cellular Uptake, Cytotoxicity and Biodistribution of Doxorubicin (DOX)-Loaded LMNs (D-LMNs)

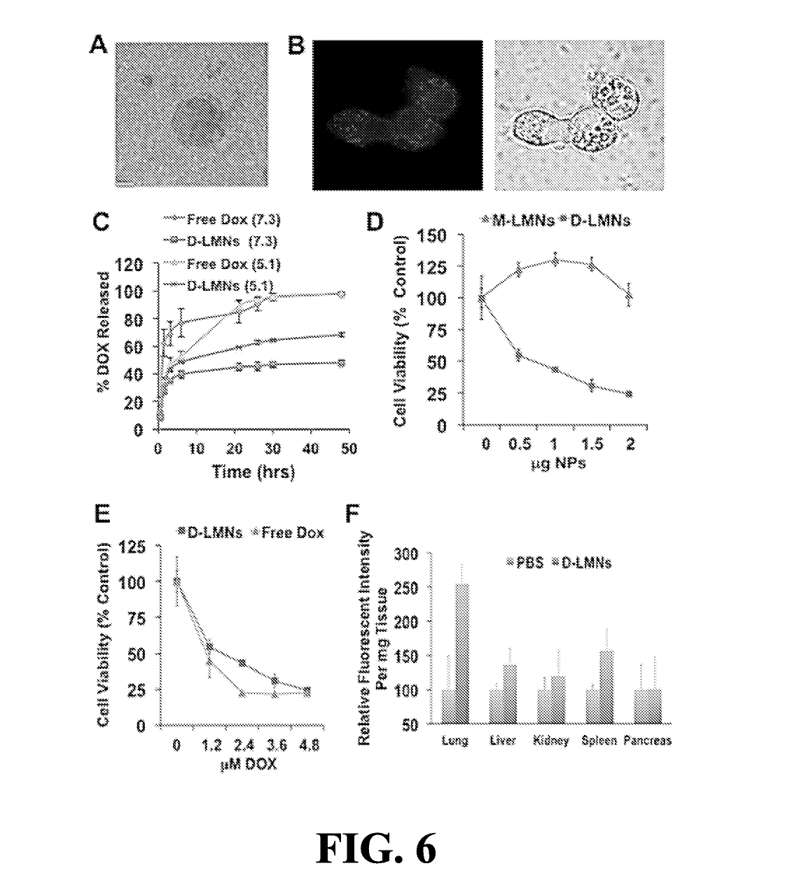

To determine the potential of LMNs to deliver small molecular drugs, MnO in the hydrophobic core was replaced with the chemotherapeutic drug DOX. More specifically, doxorubicin hydrochloride (DOX) along with 4 molar equivalents of triethylamine was added to chloroform and the mixture was sonicated for 30 minutes to dissolve the DOX. Phospholipid micelles encapsulating DOX (referred to as D-LMNs) or DOX and MnO (referred to as DM-LMNs) were prepared as previously described with some modifications [R. Kumar et al., Theranostics 2012, 714-722; Y. Namiki et al., Nature Nanotechnology 2009, 4, 598]. The 3 mg of MnO was replaced by 3 mg of DOX in D-LMNs, and in DM-LMNs, the 3 mg of MnO was replaced by 1.5 mg of DOX and 1.5 mg of MnO. D-LMNs were found to be spherical, as judged by TEM (FIG. 6A) with a hydrodynamic radius of about 100 nm and a positive zeta potential.

To evaluate the potential of these nanoparticles for therapeutic delivery, in vitro cellular uptake of D-LMNs was evaluated. Cellular uptake experiments using D-LMNs and DM-LMNs were performed in the same manner as the FM-LMNs uptake studies. Cells were seeded 24 hours prior to transfection into an 8-well chamber slide at a density of 80,000 cells per well in 300 μl of complete medium (DMEM containing 10% FBS, 2 mM L-glutamate and 1% penicillin/streptomycin). At the time of FM-LMNs addition, the medium in each well was replaced with 250 μl of fresh DMEM without FBS. Various amounts of FM-LMNs, diluted in 50 μl DMEM with no FBS, were added to each well. After 4 hours of incubation, the cells were washed with PBS and fixed with 10% neutral buffered formalin. Nuclei were stained with DAPI. The distribution of FM-LMNs inside the cells was determined with a multiphoton Olympus BX61W1 confocal microscope.

Fluorescence images of HEK293 cells incubated with D-LMNs for 24 hours showed that most of the DOX was distributed in the cytoplasm of the cell (FIG. 6B). This is in contrast to cells incubated with free DOX, where the DOX is found in the nuclei intercalated with DNA [R. Kumar et al., Theranostics 2012, 714-722]. These data suggest that the internalization mechanism of the D-LMNs is different from that of free DOX. Similar results have been reported before by other groups using micellar carriers to deliver DOX to cells [X. Shuai et al., Journal of Controlled Release: Official Journal of the Controlled Release Society 2004, 98, 415].

An in vitro DOX release study was also performed wherein D-LMNs, M-LMNs, and free DOX were each dispersed in 1 ml PBS buffer containing 1% Tween 20 (pH 7.3 or pH 5.1) and placed in a dialysis membrane (MWCO of 12000-14000 Da), The bag was then immersed in a tube containing 10 mL of the same PBS buffer (pH 7.4 or pH 5.1) and incubated at 37° C. At specific time intervals the DOX content in the PBS was analyzed using the UV-VIS spectrophotometer at 485 nm. Samples were all done in triplicate. M-LMNs were used as a blank. The release of the encapsulated DOX from D-LMNs was determined at pH 7.3, which is the physiological pH, and at pH 5.1, which represents the acidic pH inside endosomes, lysosomes, and solid tumors. One percent Tween 20 was used because it forms hydrophobic pockets, which can stabilize the released DOX from the D-LMNs, and helps to avoid aggregation of the hydrophobic DOX in the aqueous environment.

The release profile of DOX from D-MLNs is shown in FIG. 6C. DOX release occurred with an initial burst during the first 6 hours with about 50% of the DOX releasing at pH 7.3 and about 40% of the DOX releasing at pH 5.1. Subsequently, the release occurred more slowly and steadily with more than 90% of the free DOX being released into the solution at either pH after 96 hours. However, even after 48 hours only 48% and 68% of the DOX had been released from the D-LMNs at pH 7.3 and pH 5.1, respectively. These results demonstrate that the DOX is sequestered within the micelles and that the pH of the environment plays a role in DOX release from the micelles. This moderate pH-dependent release may be due to the protonation of the amine head group on DOPE in an acidic environment, which could be causing destabilization of the micelle and subsequent release of the contents [H. Farhood, N. Serbina & L. Huang, Biochimica et Biophysica Acta 1995, 1235, 289].

To determine whether D-LMNs can deliver active free DOX, LLC1 cells were incubated with D-LMNs containing various concentrations of DOX, and cell viability after 72 hours was determined. More specifically, D-LMNs, M-LMNs, and free DOX were each dispersed in 1 ml PBS buffer containing 1% Tween 20 (pH 7.3 or pH 5.1) and placed in a dialysis membrane (MWCO of 12000-14000 Da). The bag was then immersed in a tube containing 10 mL of the same PBS buffer (pH 7.4 or pH 5.1) and incubated at 37° C. At specific time intervals the DOX content in the PBS was analyzed using the UV-VIS spectrophotometer at 485 nm. Samples were all done in triplicate. M-LMNs were used as a blank. FIG. 6D shows the results of this study. The D-LMNs are just as toxic to the cells as free DOX when used at the same DOX concentrations (FIG. 6E), It is also clear that these toxic effects are due solely to the DOX and not the other components of the micelles, as M-LMNs alone exerted no cytotoxic effects (FIG. 6D). These results demonstrate that D-LMNs can deliver DOX as a payload to kill tumor cells.

To study the in vivo biodistribution and safety of D-LMNs, C57B1/6 mice were treated with six rounds of intranasal instillations of D-LMNs over a period of two weeks. More specifically, C57B1/6 mice were treated with six rounds of intranasal instillations of D-LMNs (50 μl containing 0.532 mM DOX solution) over a period of two weeks. Control mice were administered PBS. The mice were then euthanized and the lung, liver, kidney, spleen, and pancreas were collected. The biodistribution of DOX in each organ was viewed using the Xenogen IVIS-200 Optical In Vivo Imaging System. The lung, liver, and kidney were then stored in OCT and frozen at −80° C. These organs were then sectioned, stained with hematoxylin and eosin, and examined for changes in morphology. For biodistribution studies, one round of D-LMNs (50 μl containing 1.1 mM DOX solution) was administered intranasally (IN) to C57BL/6 mice. Control mice were administered PBS (IN). Twenty-four and 48 hours after the administration, lung, liver, and kidney were excised. The lung, liver, and kidney were then stored in OCT and frozen at −80° C. These organs were then sectioned and viewed using fluorescence microscopy to determine DOX uptake.

From FIG. 6F it can be seen that, when administered intranasally, the D-LMNs preferentially accumulate and release DOX in the lungs. The relatively low levels of DOX in the other organs suggest that D-LMN nanoparticles outside the lung are efficiently cleared from the body. All of these results together demonstrate the potential of D-LMNs for administering chemotherapeutic agents for the treatment of lung cancer.

To evaluate potential toxicity of the D-LMNs in vivo organ sections were stained with hematoxylin/eosin (H&E) and examined by light microscopy. It is well known that high levels of free DOX are highly toxic to tissues and can cause ulcerations and necrosis. Despite relatively high levels of DOX accumulation in the lungs, liver, and kidneys, which was confirmed by biodistribution studies (FIG. 6F), no morphological or histological alterations in the organs were observed (data not shown). The reduction of systemic toxicity can most likely be attributed to the DOX being sequestered within the cationic lipid nanoparticles and not being released in the bloodstream. These studies demonstrate that D-LMNs are able to minimize the chemotherapeutic side effects of DOX on susceptible organs.

### Example 6

### Multifunctional LMNs for Simultaneous Delivery of MnO, pDNA and DOX

To evaluate the potential of LMNs to deliver functional MnO as a T1 contrast agent, DOX for chemotherapy and plasmid DNA for gene therapy, we synthesized a multifunctional LMN incorporating a mixture of hydrophobic DOX and MnO in the core and the negatively-charged pDNA on the positively-charged surface. The resulting particles, which are referred to as DM-LMNs, had a positive zeta potential and spherical morphology with a diameter similar to M-LMNs (200 nm). To examine whether DM-LMNs were also capable of providing efficient T1 MRI contrast, the DM-LMNs were analyzed using the same MR phantom imaging as M-LMNs. At a concentration of 1 mM, DM-LMNs were able to provide a T1 relaxivity that was only slightly less than that of M-LMNs (data not shown). These results suggest that these micelles can provide effective T1 MRI contrast.

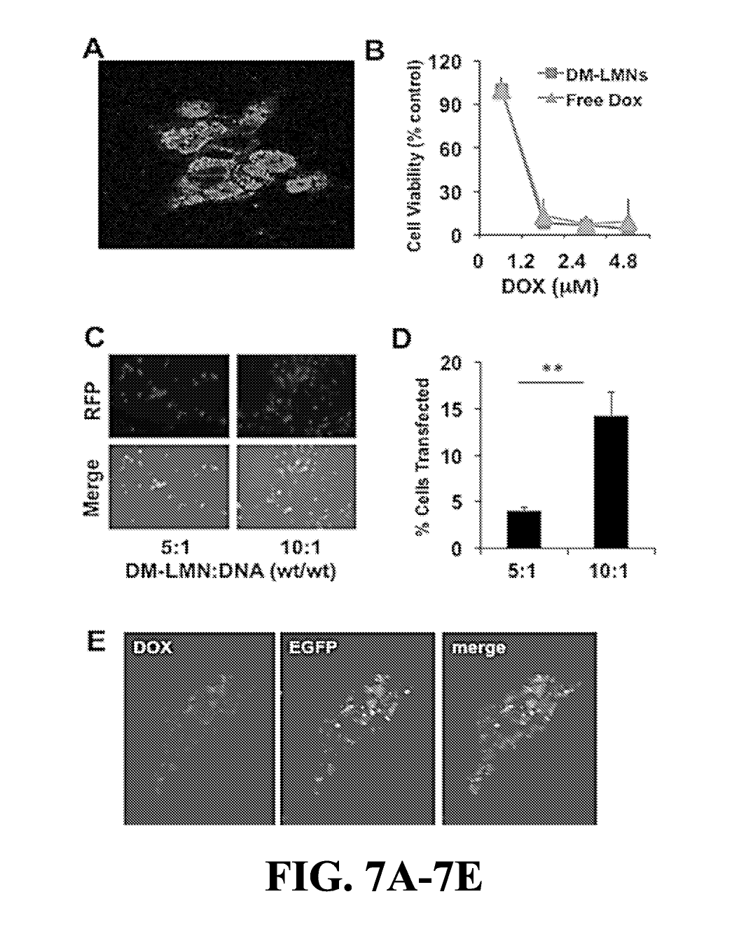

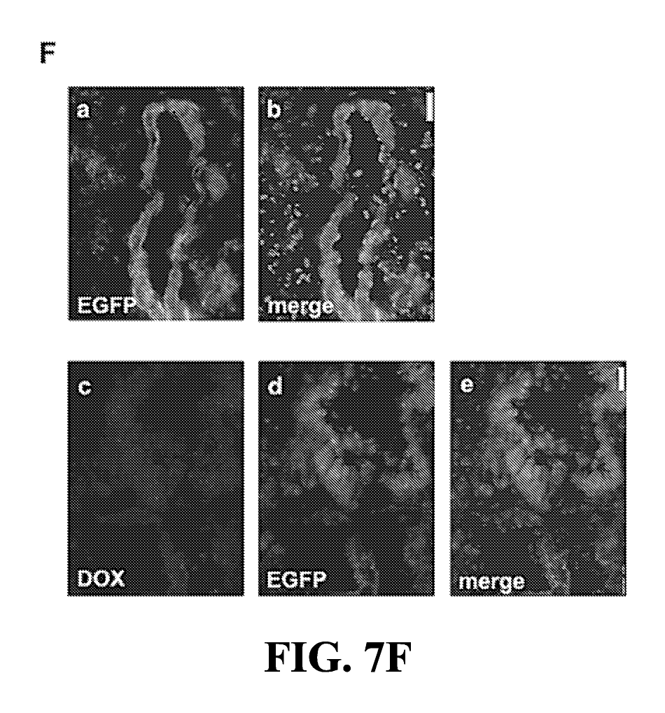

HEK293 cells were incubated with DM-LMNs for 24 hours and DOX uptake was determined by analysis of confocal fluorescence microscope images. In vitro transfections using DM-LMNs were carried out in the same manner as transfections using M-LMNs, except that the simultaneous GFP/DOX transfection was done using an 8-well chamber slide instead of a 96-well plate for imaging purposes. HEK293 cells were plated with a density of 80,000 cells per well in 300 μl of complete medium (DMEM containing 10% FBS, 2 mM L-glutamate and 1% penicillin/streptomycin). After 48 hours, the cells were fixed onto the slide using 10% neutral buffered formalin and viewed for GFP and DOX using fluorescence microscopy. Similar to D-LMNs, DOX was seen mostly in the cytoplasm of the cells (FIG. 7A). Treatment of LLC1 cells with DM-LMNs for 72 hours showed cytotoxicity comparable to that seen with LLC1 cells incubated with free DOX. With DOX concentrations of 1 μM or higher, over 50% of the cells were killed (FIG. 7B). These results show that DM-LMNs can deliver DOX as efficiently as D-LMNs while still retaining MRI contrast ability.

It was then determined whether DM-LMNs could deliver nucleic acids as efficiently as M-LMNs. HEK293 cells were transfected with DM-LMNs at the same DM-LMNs:ptd-Tomato DNA weight ratios as M-LMNs. The results (FIGS. 7C and 7D) show that DM-LMNs were capable of administering ptd-Tomato DNA to HEK293 cells with slightly less efficiency than M-LMNs. This can most likely be attributed to the loss of cells due to DOX-induced cytotoxicity. To image the simultaneous delivery of DNA and DOX, HEK293 cells were incubated with DM-LMNs complexed with DNA encoding enhanced green fluorescent protein (EGFP) for 48 hours. HEK293 cells can be seen with DOX located throughout the cytoplasm, similar to D-LMNs, and EGFP expression throughout the cytoplasm (FIG. 7E). These data show that DM-LMNs can simultaneously deliver nucleic acids and chemotherapeutic agents into cells.

To determine if DM-LMNs are capable of simultaneously administering DNA and DOX in vivo, nanoparticles complexed with EGFP-DNA were administered intranasally to mice. More specifically, C57BL/6 mice were administered 125 μg of M-LMNs complexed with 25 μg EGFP-DNA in 50 μl PBS (n=2) or 125 μg of DM-LMNs complexed with 25 μg GFP-DNA (with 0.25 mM DOX in 50 μl) (n=2) intranasally. Seventy-two and 96 hours after the administration, lung, liver, and kidney were excised. The lung, liver, and kidney were then stored in OCT and frozen at −80° C. The organs were sectioned and stained for GFP using anti-GFP antibody, then viewed for GFP expression and DOX uptake using fluorescent microscopy.

At 72 and 96 hours after administration, mice were euthanized and the lungs, liver, and kidneys were collected in OCT and frozen at −80° C. Frozen sections were immunostained for EGFP and analyzed for GFP expression and DOX uptake using fluorescence microscopy. EGFP and DOX expression could be seen in the lungs at both 72 and 96 hours (FIG. 7F). These results suggest that, regardless of the payload. LMNs are able to preferentially accumulate in the lungs when delivered intranasally and that these nanoparticles are capable of the simultaneous delivery of DNA and DOX in vivo. Taken together, these experimental results show that LMNs provide a simple and efficient theranostic micellar formulation that can be used as a multifunctional vehicle for imaging and therapy of cancer in vitro and in vivo.

1. A micelle composition comprising a micelle and either or both of a pharmaceutical core and a polynucleotide, wherein the micelle comprises a polyethylene glycol-phosphatidyl ethanolamine (PEG-PE), a DC-cholesterol, and a dioleoylphosphatidyl-ethanolamine (DOPE).

2. The micelle composition of claim 1, comprising a micelle and a polynucleotide, wherein the micelle comprises a PEG-PE, a DC-cholesterol, and a DOPE, and wherein the polynucleotide is coated onto the micelle.

3. The micelle composition of claim 2, further comprising a hydrophobic manganese-oleate core.

4. The micelle composition of claim 1, comprising a micelle and a pharmaceutical core, wherein the micelle comprises a PEG-PE, a DC-cholesterol, and a DOPE.

5. The micelle composition of claim 1, wherein the PEG has an average molecular weight of between approximately 1800 Da and 2300 Da.

6. The micelle composition of claim 5, wherein the PEG-PE, the DC-cholesterol, and the DOPE, comprise approximately 2%, 66%, and 32%, respectively, of the micelle.

7. The micelle composition of claim 1, wherein the micelle consists essentially of a PEG-PE, a DC-cholesterol, and a DOPE.

8. The micelle composition of claim 7, wherein the PEG-PE, the DC-cholesterol, and the DOPE, comprise approximately 2%, 66%, and 32%, respectively, of the micelle.

9. The micelle composition of claim 8, wherein the PEG has an average molecular weight of between approximately 1800 Da and 2300 Da.

10. A method of administering one or more compounds to a cell comprising, administering to the cell a micelle composition comprising 1) a polyethylene glycol-phosphatidyl ethanolamine (PEG-PE), a DC-cholesterol, and a dioleoylphosphatidyl-ethanolamine (DOPE), and 2) the one or more compounds, wherein the compounds are selected from the group consisting of a polynucleotide and a pharmaceutical composition.

11. The method of claim 10, wherein the PEG has an average molecular weight of between approximately 1800 Da and 2300 Da.

12. The method of claim 10, wherein the PEG-PE, the DC-cholesterol, and the DOPE, comprise approximately 2%, 66%, and 32%, respectively, of the micelle.

13. The method of claim 10, wherein the one or more compounds is a polynucleotide, and the polynucleotide is coated onto the micelle.

14. The method of claim 13, further comprising detecting the micelle composition, wherein the micelle composition further comprises a hydrophobic manganese-oleate core, and wherein the micelle composition is detected using magnetic resonance imaging technology.

15. The method of claim 10, wherein the one or more compounds is a pharmaceutical and the micelle composition comprises a pharmaceutical core.

16. The method of claim 10, wherein the one or more compounds are a polynucleotide and a pharmaceutical, and wherein the polynucleotide is coated onto the micelle and the micelle composition comprises a pharmaceutical core.

17. The method of claim 10, wherein the cell is a lung cell in a subject and the micelle composition is administered to the subject intranasally.

18. The method of claim 10, wherein the micelle consists essentially of a PEG-PE, a DC-cholesterol, and a DOPE.

19. The method of claim 18, wherein the PEG-PE, the DC-cholesterol, and the DOPE, comprise approximately 2%, 66%, and 32%, respectively, of the micelle.

20. The method of claim 19, wherein the PEG has an average molecular weight of between approximately 1800 Da and 2300 Da.
